(19) United States
(12) Patent Application Publication
(10) Pub. No.: US 2014/0065611 A1
(43) Pub. Date: Mar. 6, 2014

(54) MODIFICATION OF NEUROBEHAVIORAL EFFECTS OF MERCURY BY A GENETIC POLYMORPHISM OF COPROPORPHYRINOGEN OXIDASE IN CHILDREN

(71) Applicant: University of Washington through its Center for Commercialization, Seattle, WA (US)

(72) Inventors: James Woods, Seattle, WA (US); Diana Echeverria, Seattle, WA (US); Nicholas J. Heyer, Seattle, WA (US); Joan Russo, Seattle, WA (US)

(73) Assignee: University of Washington through its Center for Commercialization, Seattle, WA (US)

(21) Appl. No.: 13/781,516

(22) Filed: Feb. 28, 2013

Related U.S. Application Data
(60) Provisional application No. 61/604,296, filed on Feb. 28, 2012.

(51) Int. Cl. C12Q 1/68

(52) U.S. Cl. CPC C12Q 1/6883
USPC 435/6.11

(57) ABSTRACT

In one aspect, the present invention provides a method and reagents for predicting a subject's susceptibility or risk to developing a neurobehavioral deficit associated with mercury exposure. The method comprises performing an assay to determine the presence or absence of a CPOX4 polymorphism in one or both alleles of the coproporphyrinogen oxidase (CPOX) gene, and classifying the susceptibility of the subject to developing a neurobehavioral deficit associated with mercury exposure. A subject determined to possess the CPOX4 polymorphism in at least one allele of the CPOX gene is classified as having an increased susceptibility to developing at least one neurobehavioral deficit associated with mercury exposure compared to a subject with no CPOX4 polymorphisms.

### CROSS-REFERENCE TO RELATED APPLICATION

This application claims the benefit of U.S. Provisional Application No. 61/604,296, filed Feb. 28, 2012, the disclosure of which is incorporated herein by reference in its entirety.

### STATEMENT OF GOVERNMENT LICENSE RIGHTS

This invention was made with Government support under P42ES04696, P30ES07033, and R21ES019632, awarded by the National Institute of Environmental Health Sciences (NIEHS) of the National Institutes of Health (NIH). The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 40601_Seq_Final_{—}2013-02-28.txt.

The text file is 6 KB; was created on Feb. 28, 2013; and is being submitted via EFS-Web with the filing of the specification.

### BACKGROUND

The central nervous system (“CNS”) is the critical target organ of elemental mercury. Exposure to mercury, whether from seafood, dental amalgam, occupation, or pollution from coal-burning power plants, poses a risk of toxicity that can manifest in neurological and neurobehavioral deficits. Children are recognized as having heightened susceptibility to the adverse effects of environmental chemicals, as compared with adults with similar exposures. Of particular concern in this respect are possible neurological deficits associated with elemental mercury (“Hg⁰”), exposure to which may cause impairment of the developing central nervous system along with attendant personality, cognitive function, and behavioral disorders. A current major challenge is the identification of those children who may be uniquely susceptible to Hg⁰-mediated neurological deficits because of genetic predisposition.

Despite advances in understanding the role of mercury in neurological disorders and deficits, there remains a need for reliable genetic biomarkers for susceptibility to neurological and neurobehavioral deficits associated with mercury exposure. The present disclosure seeks to fulfill this need and provide further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure provides a method for predicting the susceptibility or risk of a subject to developing a neurobehavioral deficit associated with mercury exposure. The method comprises performing an assay on a biological sample obtained from a subject to determine the presence or absence of a CPOX4 polymorphism in one or both alleles of the coproporphyrinogen oxidase (CPOX) gene. The method also comprises classifying the susceptibility of the subject to developing a neurobehavioral deficit associated with mercury exposure, wherein a subject determined to possess the CPOX4 polymorphism in at least one allele of the CPOX gene is classified as having an increased susceptibility to developing at least one neurobehavioral deficit associated with mercury exposure compared to a subject with no CPOX4 polymorphisms.

In some embodiments, the subject is human. In some embodiments, the subject is male. In some embodiments, the subject is female.

In some embodiments, the subject is a young human that is about 20 years old or younger. In some embodiments, the subject is an embryo or fetus in utero.

In some embodiments, the subject possessing the CPOX4 polymorphism in both alleles of the CPOX gene is classified has having an increased susceptibility to developing at least one neurobehavioral deficit associated with mercury exposure compared to a subject with one or no CPOX4 polymorphisms.

In some embodiments, the at least one neurobehavioral deficit negatively affects performance in at least one neurobehavioral domain selected from the group of domains consisting of the intelligence domain, attention domain, visual-spatial domain, executive functioning domain, learning and memory domain, and motor domain. In some embodiments, the performance in the attention domain can be determined by the Stroop test, the WAIS III Digit Span test, or the WMS III Spatial Span test. In some embodiments, performance in the visual-spatial domain can be determined by the Simple Reaction Time test or the WAIS III test. In some embodiments, performance in the executive functioning domain can be determined by the Wisconsin Card Sort test or the Adult Trials B test. In some embodiments, performance in the learning and memory domain can be determined by the RAVALT test or the WMS III—Visual Reproductions test. In some embodiments, performance in the motor domain can be determined by the WRAVMA—Pegs test or the Finger Tapping test.

In some embodiments, a reduction in performance in at least one neurobehavioral domain in a subject possessing the CPOX4 polymorphism in at least one allele of the CPOX gene and exposed to mercury is greater than the added reductions in performance in the same neurobehavioral domain observed for 1) a subject with no CPOX4 polymorphisms and exposed to mercury, and 2) a subject with the same number of CPOX4 polymorphisms with no exposure to mercury.

In some embodiments, a subject possessing the CPOX4 polymorphism in at least one allele of the CPOX gene has an increased susceptibility to developing a neurobehavioral deficit with exposure to a low level of mercury exposure. In some embodiments, the low level of mercury exposure is an exposure to mercury in an amount sufficient to result in a detectable urinary mercury level of 5 μg/g creatinine or less. A subject possessing the CPOX4 polymorphism in at least one allele of the CPOX gene can have an increased susceptibility to developing a neurobehavioral deficit with an acute or chronic mercury exposure.

In some embodiments, the subject possessing the CPOX4 polymorphism in at least one allele of the CPOX gene has an increased susceptibility to developing a neurobehavioral deficit within about one month of a mercury exposure.

In some embodiments, the biological sample contains genomic DNA. In some embodiments, the method further comprises detecting the presence of an adenine (A) to cytosine (C) change in exon 4, relative to the wild type CPOX gene, resulting in an asparagine (Asp, N) to histidine (His, H) substitution at amino acid position 272 of the corresponding CPOX polypeptide.

In some embodiments, the method further comprises performing an assay on a second biological sample obtained from the subject to determine the exposure status of the subject to mercury. In some embodiments, the second biological sample comprises urine, blood, serum, plasma, or hair. In some embodiments, the assay performed on the second biological sample is cold vapor atomic fluorescence spectrometry or inductively coupled plasma mass spectrometry.

In some embodiments, a subject determined to have an increased susceptibility of developing at least one neurobehavioral deficit associated with mercury exposure is advised to reduce ongoing or future risk of mercury exposure.

### DETAILED DESCRIPTION

The following description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure. Therefore, unless defined otherwise, all technical and scientific terms used herein have the meaning commonly understood by one of ordinary skill in the art to which this invention belongs.

Unless the context clearly requires otherwise, throughout the description and the claims, the words “comprise,” “comprising,” and the like are to be construed in an inclusive sense as opposed to an exclusive or exhaustive sense; that is, in the sense of “including, but not limited to.” Words using the singular or plural number also include the plural or singular number, respectively. Additionally, the words “herein,” “above,” and “below,” and words of similar import, when used in this application, shall refer to this application as a whole and not to any particular portions of this application.

The present disclosure addresses the susceptibility or risk of a subject to developing at least one neurobehavioral deficit associated with mercury exposure. Individuals are often subject to low exposures of environmental toxins, such as mercury. Exposures to mercury in its various forms can occur as a result of consumption of seafood, the use of dental amalgams, occupation or residential hazards, and exposure to pollution from coal-burning power plants. Such exposures can vary widely in terms of the resulting impacts, which can range from unobservable to significant neurobehavioral deficits that are quantifiable using techniques such as those described herein. Individuals can vary in their susceptibility to the negative impacts of mercury exposure, with some individuals possessing a mercury susceptibility phenotype. A “mercury-susceptibility phenotype” is a phenotype in an individual that displays a predisposition towards developing one or more neurobehavioral deficits after exposure to mercury. In some embodiments, the mercury susceptibility phenotype is further defined as an individual that displays a predisposition towards developing one or more neurobehavioral deficits after exposure to low levels of mercury. A phenotype that displays a predisposition for mercury sensitivity, can, for example, show a higher likelihood that one or more neurobehavioral deficits will develop in an individual with the phenotype after exposure to mercury than in members of a relevant general population under a given set of environmental conditions (diet, physical activity regime, geographic location, etc.) after exposure to the same level of mercury. Alternatively, the predisposition might show a likelihood of developing a more significant or severe neurobehavioral deficit after exposure to mercury than in members of the relevant population. Mercury-susceptibility phenotypes are believed to have some genetic component, wherein the subject with the phenotype has a genetic variation that underlies the susceptibility. The genetic variation can be used to identify or predict the subject's susceptibility to the negative impacts associated with mercury exposure.

Studies of the neurotoxic effects of elemental mercury (“Hg⁰”) in adults have identified at least four commonly expressed genetic polymorphisms that modify the effects of Hg⁰ on a wide range of neurobehavioral functions (Echeverria, D., et al., “Chronic Low-Level Mercury Exposure, BDNF Polymorphism, and Associations With Memory, Attention and Motor Function,” Neurotox. Teratol. 27:781-796, 2005; Echeverria, D., et al., “The Association Between a Genetic Polymorphism of Coproporphyrinogen Oxidase, Dental Mercury Exposure, and Neurobehavioral Response in Humans,” Neurotox. Teratol. 28:39-48, 2006; Echeverria, D., et al., “The Association Between Serotonin Transporter Gene Promoter Polymorphism (5-HTTLPR) and Elemental Mercury Exposure on Mood and Behavior in Humans,” J. Toxicol. Environ. Health 73:552-569, 2010; Heyer, N. J., et al., “Chronic Low-Level Mercury Exposure, BDNF Polymorphism and Associations With Self-Reported Symptoms and Mood,” Toxicol. Sci. 81:354-363, 2004; Heyer, N. J., et al., “The Association Between Serotonin Transporter Gene Promoter Polymorphism (5-HTTLPR), Self-Reported Symptoms, and Dental Mercury Exposure,” J. Toxicol. Environ. Health 71:1318-26, 2008; Heyer, N. J., et al., “Catechol-O-Methyltransferase (COMT) Val158Met Functional Polymorphism, Dental Mercury Exposure, and Self-Reported Symptoms and Mood,” J. Toxicol. Environ. Health 72:599-609, 2009). Of particular interest in this respect is a single nucleotide polymorphism (“SNP”) consisting of an adenine (A) to cytosine (C) change (“A>C”) in exon 4 of the gene encoding the heme biosynthetic pathway enzyme, coproporphyrinogen oxidase (“CPOX,” see standard enzyme designation EC 1.3.3.3). See Genbank SNP reference identifier rs1131857. This A>C SNP, referred to herein as “CPOX4,” encodes an asparagine-to-histidine residue change at amino acid position 272 (“N272H”) of the CPOX polypeptide, which both increases sensitivity to the neurobehavioral effects of Hg⁰ in adults (Echeverria, D., et al., “The Association Between a Genetic Polymorphism of Coproporphyrinogen Oxidase, Dental Mercury Exposure, and Neurobehavioral Response in Humans,” Neurotox. Teratol. 28:39-48, 2006) and modifies urinary porphyrin excretion as a potential biomarker of this effect in adults (Woods, J. S., et al., “The Association Between Genetic Polymorphisms of Coproporphyrinogen Oxidase and an Atypical Porphyrinogenic Response to Mercury Exposure in Humans,” Toxicol. Appl. Pharmacol. 206:113-120, 2005; Li, T., and J. S. Woods, “Cloning, Expression, and Biochemical Properties of CPOX4, a Genetic Variant of Coproporphyrinogen Oxidase That Affects Susceptibility to Mercury Toxicity in Humans,” Toxicol. Sci. 109:228-236, 2009). The population frequencies of the homozygous wild type (A/A), heterozygous (A/C) and homozygous mutant (C/C) genotypes within this cohort were 0.72, 0.25, and 0.03, respectively, and were equally prevalent among males and females, suggesting substantial exposure to the CPOX4 variant. The presence of a CPOX4 allele in combination with Hg⁰ exposure was found to result in an additive increase in sensitivity to the neurobehavioral effects of Hg⁰ in adults.

As described above, elemental mercury (“Hg⁰”) is neurotoxic. Children may be particularly susceptible to the neurotoxic effects of Hg⁰, considering that the brain and other components of the nervous system continue to develop through adolescence. Accordingly, a current major challenge is the identification of children who may be uniquely susceptible to Hg⁰ toxicity because of genetic disposition. The methods disclosed herein provide a valuable tool to assess subjects for the susceptibility or risk of developing neurobehavioral deficits upon a potential or hypothetical exposure to mercury. The methods can be used to reduce or prevent neurobehavioral deficits associated with a potential future fig exposure by identifying high-risk individuals and thereby promoting adjustments to their behavior, environment, or even medical and dental care, so as to moderate their likelihood of mercury exposure.

In the study described in more detail below, the role of the CPOX4 allele on the adverse neurobehavioral effects of Hg⁰ exposure in children was examined. Subjects were children and adolescents who participated in a recently completed prospective randomized dental amalgam clinical trial between ages 8-18 and for whom longitudinal (annual) neurobehavioral assessments and quantitative measures of dental amalgam Hg⁰ exposure over 7 years of follow-up were available. Specifically, 507 children, 8-12 years of age at baseline, participated in the clinical trial. Subjects were evaluated at baseline and at 7 subsequent annual intervals for neurobehavioral performance and urinary mercury levels. Following the completion of the clinical trial, genotyping assays for CPOX4 allelic status were performed on biological samples provided by 330 of the trial participants. Regression modeling strategies were employed to evaluate associations between CPOX4 status, Hg⁰ exposure, and neurobehavioral test outcomes.

Additionally, to preclude selection bias possibly associated with those genotyped for CPOX4 per se, comparable assessments were made with respect to second single nucleotide polymorphism located at exon 5 encoding a guanine (G) to adenine (A) change (“G>A”) of the CPOX gene encoding a synonymous mutation at amino acid residue position 330 in the CPOX enzyme (E330E). See Genbank SNP reference identifier rs1729995. This SNP allele is referred to herein as “CPOX5”. CPOX5 has been previously identified as distributed among men and women within the adult dental population with frequencies of the homozygous common (wild type), heterozygous, and homozygous mutant alleles of 0.48, 0.43 and 0.09, respectively (Woods, J. S., et al., “The Association Between Genetic Polymorphisms of Coproporphyrinogen Oxidase and an Atypical Porphyrinogenic Response to Mercury Exposure in Humans,” Toxicol. Appl. Pharmacol. 206:113-120, 2005). CPOX5 is not known to be in linkage disequilibrium with CPOX4. These assessments were made independently in boys and girls.

Among girls, some significant CPOX4-Hg⁰ interactions or independent main effects for Hg⁰ or CPOX4 were observed. However, among boys, numerous significant interaction effects between CPOX4 and Hg⁰ were observed spanning all 5 domains of neurobehavioral performance. All underlying dose-response associations between Hg⁰ exposure and test performance were restricted to boys with the CPOX4 variant, and all of these associations were in the impaired direction where increased exposure to Hg⁰ decreased performance. These findings are the first to demonstrate genetic susceptibility to the adverse neurobehavioral effects of Hg⁰ exposure in children, and moreover demonstrate a synergistic interaction of CPOX4 status with Hg⁰ exposure on the neurobehavioral effects of Hg⁰ exposure in children. The relative paucity of responses among girls compared with same-age boys with comparable Hg⁰ exposure provides evidence of sexual dimorphism in genetic susceptibility to the adverse neurobehavioral effects of Hg⁰ in children and adolescents.

In accordance with the foregoing, in one aspect, the disclosure provides a method for predicting the susceptibility or risk of a subject to developing a neurobehavioral deficit associated with mercury exposure. The method comprises determining the presence or absence of a CPOX4 polymorphism in one or both alleles of the coproporphyrinogen oxidase (CPOX) gene. The method also comprises classifying the susceptibility or risk of the subject to developing a neurobehavioral deficit associated with mercury exposure. When a subject is determined to have the CPOX4 polymorphism in at least one allele of the CPOX gene, the subject is classified as having an increased susceptibility or risk of developing a neurobehavioral deficit associated with mercury exposure as compared to a subject with no CPOX4 polymorphisms.

The susceptibility to or risk of a subject developing a neurobehavioral deficit associated with mercury exposure is classified based on the subject's CPOX allele status, specifically, whether the subject possess at least one CPOX4 single nucleotide polymorphism in the coproporphyrinogen oxidase (CPOX) gene. A nucleic acid polymorphism is characterized by two or more different “alleles,” or versions of the nucleic acid sequence, in this case, the CPOX gene. Typically, an allele of a polymorphism that is identical to a reference sequence is referred to as a “reference” or “wild type” allele, and an allele of a polymorphism that is different from a reference sequence is referred to as an “alternate” allele, or sometimes a “variant” allele. Usually, the “reference” or “wild type” allele is the more frequently occurring allele at a given polymorphic site, and the “alternate” or “variant” allele is the less frequently occurring allele, as present in the general or study population. The term “single nucleotide polymorphism” or “SNP” refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site.

Coproporphyrinogen oxidase (CPOX) (EC 1.3.3.3) is the sixth enzyme in the heme biosynthetic pathway and catalyzes the oxidative decarboxylation of the propionic acid side chains on rings A and B of coproporphyrinogen-III to vinyl groups, producing protoporphyrinogen-IX. The CPOX protein contains a number of reduced thiol residues that render it potentially susceptible to inhibition by thiol binding agents, including metals. The human gene encoding the CPOX enzyme is located at cytogenic map location 3q12 on the third chromosome. The sequence for the gene, including non-coding regions, is available in the National Center for Biotechnology Information's (NCBI's) Genbank database at reference no. NG_{—}015994, last accessed Feb. 19, 2013. The reverse-transcribed sequence of the CPOX gene (i.e., derived from the mRNA sequence and, thus, is without introns) is available in NCBI Genbank at reference sequence no. NM_{—}000097.5, last accessed Feb. 19, 2013, and is also set forth herein as SEQ ID NO:1. The CPOX4 SNP is a missense substitution appearing in exon 4 of the CPOX gene wherein an adenine (A) is replaced with a cytosine (C). With reference to the genomic sequence, i.e., Genbank sequence no. NG_{—}015994, exon 4 spans from nucleotide positions 9758 to 9899. The CPOX4 A>C substitution appears at nucleotide position 9760 (listed in the reference Genbank sequence no. NG_{—}015994 as an adenine). With reference to the reverse-transcribed sequence, Genbank sequence no. NM 000097.5, exon 4 corresponds to nucleotide positions 919 to 1060. The CPOX4 A>C substitution appears at nucleotide position 921 (listed in the reference Genbank sequence no. NM_{—}000097.5 as an adenine). The sequence of the CPOX4 SNP is available in the Genbank database as SNP reference no. rs1131857, which includes a portion of the non-coding sequence immediately upstream of the sequence encoding exon 4 starting at nucleotide position 9734 of Genbank sequence no. NG_{—}015994. The specific location of the CPOX4 A>C SNP is at nucleotide position 27. The sequence is also set forth herein as SEQ ID NO:2, wherein the SNP at nucleotide position 27 is represented therein as an N, wherein N can be an A (as in the wild type) or C (as in CPOX4 SNP). The CPOX4 SNP results in an asparagine (Asp, N) to histidine (His, H) substitution at amino acid position 27 in the encoded CPOX protein.

Determining a subject's CPOX allele status, for example, detecting the presence of one or more CPOX4 alleles, can be accomplished using any method known in the art, for example, Southern or northern analyses, in situ hybridization analyses, single-stranded conformational polymorphism analyses, polymerase chain reaction (PCR) analyses and nucleic acid microarray analyses, all of which are well known to those of skill in the art. “Hybridization-based” assays rely on the formation of a stable duplex or triplex between a probe and a target nucleotide sequence for detecting or measuring such a sequence. Hybridization-based assays include, without limitation, assays based on the use of oligonucleotides, such as polymerase chain reactions, oligonucleotide ligation reactions, single-base extensions of primers, circularizable probe reactions, allele-specific oligonucleotide hybridizations, either in solution phase or bound to solid phase supports, such as microarrays or microbeads.

In one example, a prognosis is made using a test sample containing genomic DNA or RNA obtained from an individual to be tested. The test sample can be from any source which contains genomic DNA or RNA, including for example, blood, amniotic fluid, cerebrospinal fluid, skin, muscle, buccal or conjunctival mucosa, placenta, gastrointestinal tract, or other tissues. Alternatively, a test sample of DNA from a fetus, fetal cells, or tissue can be obtained by appropriate methods such as by amniocentesis, chorionic villus sampling, or by sequencing trace fetal DNA appearing in the mother's blood, as are known in the art. The test sample is subjected to one or more tests to identify the presence or absence of the CPOX4 allele (which is predictive of mercury sensitivity).

In one embodiment, the test sample is subjected to purification, isolation and/or amplification techniques, many of which are well known in the art (e.g., Sambrook et al. (1989) Molecular Cloning, A Laboratory Manual, Cold Spring Harbor Press, N.Y.; and Ausubel et al. (eds. 1987-1993), Current Protocols in Molecular Biology, John Wiley & Sons, Inc. New York). The terms “isolated” and “purified” refer to a material, e.g., nucleic acids, that is substantially or essentially removed from or concentrated in its natural environment. For example, a purified nucleic acid is one that is separated from the nucleic acids that normally flank it or from other biological materials (e.g., other nucleic acids, proteins, lipids, cellular components, etc.) in a sample.

In one embodiment, Southern blot, northern blot or similar analyses, methods are used to identify the presence or absence of one or more genomic DNA sequences associated with resistance or susceptibility to Hg using complementary nucleic acid probes. In certain embodiments, the nucleic acid probes have detectable labels attached thereto before they are contacted with the test sample; in other embodiments, the nucleic acids in the test sample have detectable labels attached thereto before they are contacted with the nucleic acid probes. The term “detectable label” as used herein refers to, for example, a luminescent label, a light scattering label or a radioactive label, or any other form of labeling that can be detected by a physical, chemical, or a biological process. Fluorescent labels include commercially available fluorescein phosphoramidites such as Fluoreprime (Pharmacia), Fluoredite (Millipore), and FAM (ABI).

Alternative diagnostic and prognostic methods employ amplification of target nucleic acids associated the CPOX4 SNP, e.g., by PCR. This is especially useful for target nucleic acids present in very low quantities. In one embodiment, amplification of target nucleic acids associated with the CPOX4 SNP indicates its presence and is a prognostic of susceptibility to Hg toxicity.

Microarrays can also be utilized for analyzing the presence of various CPOX alleles. Microarrays comprise probes that are complementary to target nucleic acid sequences from an individual. A microarray probe is preferably allele-specific, such as CPOX4 specific. In one embodiment, the microarray comprises a plurality of different probes, each coupled to a surface of a substrate in different known locations and each, capable of binding complementary strands. See, e.g., U.S. Pat. No. 5,143,854 and PCT Publication Nos. WO 90/15070 and WO 92/10092. These microarrays can generally be produced using mechanical synthesis methods or light-directed synthesis methods that incorporate a combination of photolithographic methods and solid phase oligonucleotide synthesis methods. See Fodor et al., (1991) Science 251:767-777; and U.S. Pat. No. 5,424,186. Techniques for the mechanical synthesis of microarrays are described in, for example, U.S. Pat. No. 5,384,261.

Other methods to detect polymorphic nucleic acids include, for example, direct manual sequencing (Church and Gilbert, (1988) Proc. Natl. Acad. Sci. USA 81:1991-1995; Sanger, F., et al. (1977) Proc. Natl. Acad. Sci. USA 74:5463-5467; and U.S. Pat. No. 5,288,644); automated fluorescent sequencing; single-stranded conformation polymorphism assays; clamped denaturing gel electrophoresis; denaturing gradient gel electrophoresis (Sheffield, V. C., et al. (1981) Proc. Natl. Acad. Sci. USA 86:232-236), mobility shift analysis (Orita, M., et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; Tm-shift genotyping (Germer et al. (1999) Genome Research 9:72-78); kinetic PCR (Germer et al. (2000) Genome Research 10:258-266); chemical mismatch cleavage (Cotton et al. (1985) Proc. Natl. Acad. Sci. USA 85:4397-4401); RNase protection assays (Myers, R. M. et al. (1985) Science 230:1242); and use of polypeptides which recognize nucleotide mismatches, such as the E. coli mutS protein. For example, as referenced below, oligonucleotide sequences useful for PCR amplification and sequencing of exon 4 of the CPOX gene are disclosed in Woods, J. S., et al., “The Association Between Genetic Polymorphisms of Coproporphyrinogen Oxidase and an Atypical Porphyrinogenic Response to Mercury Exposure in Humans,” Toxicol. Appl. Pharmacol. 206:113-120, 2005, and are set forth herein as SEQ ID NO:3 (sense primer: 5′-AAGTCCACATTAGAATCCC-3′) and SEQ ID NO:4 (antisense primer: 5′-GTTGCCTTCAGAAGGAACAG-3′).

Additionally, as described below, the inventors used a fluorescence 5′-nuclease assay to detect the presence of a wild type or CPOX4 allele in the CPOX gene utilizing labeled probes specific for each allele as it appears in the genomic DNA (thus, including sequence complementary to noncoding regions flanking exon 4 region). The probe sequence specific for the wild type CPOX allele is set forth herein as SEQ ID NO:5 (i.e., 5′-FAM-ACCACTGCTTGTTGCCTACCAAATCA-TAMRA-3′; the reverse complement sequence; “FAM” and “TAMRA” are the probe label/quencher dyes; the SNP site is underlined). The probe sequence specific for the wild type CPOX allele is set forth herein as SEQ ID NO:6 (i.e., 5′-TET-ACCACTGCTTGTGCCTACCAAATC-TAMRA-3V; the reverse complement sequence; “FAM” and “TAMRA” are the probe label/quencher dyes; the SNP site is underlined). Primers used to amplify the genomic template including the SNP site are set forth herein as SEQ ID NO:7 (forward: 5′-CCAGTAATGCTGAATCTCAAAAGTCC-3′) and SEQ ID NO:8 (reverse: 5′-GGACAGCGTCTTCTTGATTCAAGTAT-3′). Intact allele-specific probes have a reporter dye, TET, and a quencher dye, TAMRA, in close proximity, thus preventing a detectable signal. After the allele-specific primers specifically hybridize to the genomic template (either wild type or CPOX4 SNP allele), the amplification process results in degradation of the annealing allele-specific probes by virtue of the 5′ nuclease activity of the enzyme. Degradation allows the reporter dye to disperse from the close proximity of the quencher, thus producing a detectable signal. It will be readily apparent to a person of ordinary skill that alternative assays using RNA or cDNA as the initial template require different probe/primer oligonucleotide sequences to allow for the absence of non-coding regions of the genomic sequence. Such probe/primer oligonucleotide sequences will ideally be based on the sequence set forth herein as SEQ ID NO:1, which is the reverse-transcribed sequence of the CPOX gene (i.e., from mRNA, and thus without introns) and is represented in the NCBI Genbank database by reference sequence no. NM_{—}000097.5.

In some embodiments, a subject determined to possess the CPOX4 polymorphism in at least one allele of the CPOX gene is classified as having an increased susceptibility to developing at least one neurobehavioral deficit associated with mercury exposure as compared to a subject with no CPOX4 polymorphisms. In some embodiments, a subject determined to possess the CPOX4 polymorphism in one allele of the CPOX gene is classified as having an increased susceptibility to developing at least one neurobehavioral deficit associated with mercury exposure as compared to a subject with no CPOX4 polymorphisms. In some embodiments, a subject determined to possess the CPOX4 polymorphism in both alleles of the CPOX gene is classified as having an increased susceptibility to developing at least one neurobehavioral deficit associated with mercury exposure as compared to a subject with no CPOX4 polymorphisms. In some embodiments, a subject determined to possess the CPOX4 polymorphism in both alleles of the CPOX gene is classified as having an increased susceptibility to developing at least one neurobehavioral deficit associated with mercury exposure, as compared to a subject with a CPOX4 polymorphisms in only one (not both) alleles of the CPOX gene, or as compared to a subject with no CPOX4 polymorphisms. In some embodiments, the subject's susceptibility or risk is compared with the susceptibility or risk of a (control) subject, wherein the control subject would be potentially or hypothetically exposed to the same or generally equivalent level of mercury.

In preferred embodiments of the method, the subject is a human. As described above, the nervous system in humans continues to develop throughout childhood and adolescence. The continuing developmental process can make the nervous system more susceptible to adverse effects of environmental toxins, such as mercury. Accordingly, in further embodiments of the method, the human subject is a young human, such as 20 years old or younger. For example, the human subject is of an age of about 1, 2, 3, 4, 5, 6, 7, 8, 9, 10, 11, 12, 13, 14, 15, 16, 17, 18, 19, or 20 years old at the time the subject is assessed for the presence of a CPOX4 polymorphism in at least one allele of the CPOX gene. In other embodiments, the subject is an embryo or fetus that is tested for the presence of a CPOX4 polymorphism in at least one allele of the CPOX gene while in utero.

The disclosure provides methods for predicting the susceptibility or risk of a subject to developing any neurobehavioral deficit associated with mercury exposure. A neurobehavioral deficit can be defined as a below-normal performance by the subject in any quantifiable neurobehavioral aspect, as understood in the art. Neurobehavioral deficits can be categorized and assessed according to any standard accepted in the art. A person of ordinary skill in the art would recognize that neurobehavioral function (and, therefore, deficits thereof) can be categorized into various domains, including an attention domain, a visual-spatial domain, an executive functioning domain, a learning and memory domain, and a motor domain. Accordingly, neurobehavioral performance of a subject is capable of assessment and comparison against an accepted norm or standard, and thus neurobehavioral deficits can be established according to art recognized protocols. For example, a subject's function or performance within any such domain can be assessed through rigorous and quantitative approaches that are practiced in the art. See, e.g., Martins, I. P., et al. “Age and Sex Difference in Neurobehavioral Performance: a Study of Portuguese Elementary School Children,” Int. J. Neurosci. 115:1687-1709, 2005. For example, performance in the attention domain can be determined by the application of various accepted tests, including the Stroop test, the WAIS III—Digit Span test, or the WMS III—Spatial Span test. Performance in the visual-spatial domain can be determined by the application of tests such as the Simple Reaction Time test or the WAIS III test. Performance in the executive functioning domain can be determined by the application of tests such as the Wisconsin Card Sort test or the Adult Trials B test. Performance in the learning and memory domain can be determined by tests such as the RAVALT test or the WMS III—Visual Reproductions test. Performance in the motor domain can be determined by tests such as the WRAVMA—Pegs test or the Finger Tapping test.

Below-normal neurobehavioral performances are capable of being determined using any test where the subject achieves a score that is less than a standard or threshold value. It will be understood by persons of skill in the art that the standard or threshold value can reflect the performance of one or more subjects considered to be neurotypical or behavioraltypical. In some embodiments, the one or more (control) subjects considered to be neurotypical or behavioraltypical would preferably be of an equivalent age of the (test) subject. The phrase “an equivalent age” is used to convey that, at the time the control subject(s) are assessed for neurobehavioral performance to determine the reference standard or threshold value, the control subject(s) is/are of an age that is similar to the age of the (test) subject at the time the test subject is assessed for neurobehavioral performance. For example, at a neurobehavioral performance assessment, the control subject's age (or subjects' ages) can be within at least 6 months to 3 years from the age of the (test) subject's age (such as within at least 6 months to at least 2 years, or within at least 6 months to 1 year) at the time of a neurobehavioral performance assessment of the (test) subject. For example, in some embodiments, at the time of a neurobehavioral performance assessment, the control subject(s) is/are of an age that is within 2 years (older or younger) of the age of the (test) subject at the time of a neurobehavioral performance assessment of the (test) subject.

In some embodiments, the one or more subjects considered to be neurotypical or behavioraltypical for purposes of determining a reference standard or threshold value for neurobehavioral performance can be determined to have no CPOX4 polymorphisms in either allele of the CPOX gene. In some embodiments, the one or more subjects considered to be neurotypical or behavioraltypical for purposes of determining a reference standard or threshold value for neurobehavioral performance have no CPOX4 polymorphisms and have are determined to have had exposure, or risk of exposure, to similar levels of mercury as for the test subject. In some embodiments, the one or more subjects considered to be neurotypical or behavioraltypical for purposes of determining a reference standard or threshold value for neurobehavioral performance have had no significant exposures to neurotoxins, such as heavy metals including mercury, regardless of genetic-based susceptibility to heavy metal toxins. In some embodiments, the standard or threshold value is obtained from or reflects the performance of one subject that is considered to be neurotypical or behavioraltypical.

In some embodiments, the standard or threshold value is obtained from or reflects the performance of a plurality of subjects that are considered to be neurotypical or behavioraltypical. For example, the reference standard or threshold value is obtained from about 2 to about 10, from about 10 to about 20, from about 20 to about 30, from about 30 to about 40, from about 40 to about 50, from about 50 to about 60, from about 60 to about 70, from about 70 to about 80, from about 80 to about 90, from about 90 to about 100 or more subjects that are neurotypical or behavioraltypical.

In some embodiments, the method is used for predicting the susceptibility or risk of a subject to developing one or more neurobehavioral deficit associated with mercury exposure. For example, the method can predict the susceptibility or risk of a subject to developing a neurobehavioral deficit in one, two, three, four, five, six, seven, and/or eight or more distinctly defined neurobehavioral domains as defined in the art, such as those described above.

In some embodiments, the increased susceptibility to or risk of developing a neurobehavioral deficit associated with mercury exposure is an increased potential to develop or manifest at least one observable neurobehavioral deficit. The increased potential to developing a neurobehavioral deficit can be expressed as an increased statistical likelihood of developing or manifesting at least one observable neurobehavioral deficit, such as about 1 to about 5, about 5 to about 10, about 10 to about 15, about 15 to about 20, about 20 to about 25, about 25 to about 30, about 30 to about 35, about 35 to about 40, about 40 to about 45, about 45 to about 50, about 50 to about 55, about 55 to about 60, about 60 to about 65, about 65 to about 70, about 70 to about 75, about 75 to about 80, about 80 to about 85, about 85 to about 90, about 90 to about 95, and about 95 to about 100 percent increased likelihood of developing or manifesting at least one observable neurobehavioral deficit as compared to a control subject, or any combination or subcombination of likelihoods, or ranges of likelihoods, recited therein. The term “about” is used herein to denote a variation of 1 to 2 percentage points above or below the stated likelihood.

In other embodiments, the increased susceptibility to or risk of developing a neurobehavioral deficit associated with mercury exposure is the susceptibility to or risk of manifesting a markedly greater neurobehavioral deficit as compared to any deficit manifested in a control subject or subjects (e.g., with no CPOX4 polymorphisms) that is/are potentially exposed to the same level of mercury. In such embodiments, it is preferred that the one or more deficits in the test subject and control are capable of being quantified using the same or equivalent techniques, such as any of those described herein, to assess neurobehavioral function in any domain.

In some embodiments, a significantly greater neurobehavioral deficit is established when the presence of at least one CPOX4 variant allele has a statistical interaction with the factor of mercury exposure. Accordingly, the neurobehavioral deficit, or reduction in performance in at least one neurobehavioral domain, observed in a subject possessing the CPOX4 polymorphism in at least one allele of the CPOX gene and who is also exposed to mercury is greater than the added reductions in performance in the same neurobehavioral domain observed for 1) a control subject with no CPOX4 polymorphisms and exposed to mercury, and 2) a subject with the same number of CPOX4 polymorphisms with no exposure to mercury. Stated otherwise, the neurobehavioral deficit associated with mercury exposure in a subject with at least one CPOX4 allele is observed to be a synergistic (i.e., better than additive) effect compared to the independent factors of at least one CPOX4 allele and mercury exposure. In some embodiments, the subjects with mercury exposure being compared have exposures to equivalent or similar levels of mercury. In some embodiments, the subjects being compared are of equivalent age.

The present inventors demonstrate herein that the presence of at least one CPOX4 variant allele in boys had a significant interaction with mercury exposure (i.e., more than an additive effect) on all 5 assayed neurobehavioral domains. Accordingly, in some embodiments of the method, the subject is male. However, the inventors also demonstrate herein that the presence of at least one CPOX4 variant allele had significant interaction with the effects of acute mercury exposure in girls as measured in two different tests of neurobehavioral function. Furthermore, the presence of at least one CPOX4 variant allele had significant main effects associated with mercury exposure in girls. Thus, in other embodiments of the method, the subject is female.

In some embodiments, the subject possessing the CPOX4 polymorphism in at least one allele of the CPOX gene has an increased susceptibility to developing a neurobehavioral deficit at any time after a potential mercury exposure, such as immediately after a potential exposure, to a period of years after a potential exposure. In some embodiments, the subject possessing the CPOX4 polymorphism in at least one allele of the CPOX gene has an increased susceptibility to developing a neurobehavioral deficit that is capable of being observed or detected as described herein within less than 24 hours, between 1-5 days, 5-10 days, 10-15 days, 15-20 days, 20-25 days, 25-30 days, or after 1 month, 2 months, 3 months, 4 months, 5 months, 6 months, 1 year, 2 years, or more, after any potential mercury exposure. In one embodiment, the subject possessing the CPOX4 polymorphism in at least one allele of the CPOX gene has an increased susceptibility to developing a neurobehavioral deficit that is capable of being observed or detected within 2 years of exposure. In one embodiment, the subject possessing the CPOX4 polymorphism in at least one allele of the CPOX gene has an increased susceptibility to developing a neurobehavioral deficit that is capable of being observed or detected within 6 months after exposure. In one embodiment, the subject possessing the CPOX4 polymorphism in at least one allele of the CPOX gene has an increased susceptibility to developing a neurobehavioral deficit that is capable of being observed or detected immediately after (i.e., within hours of) exposure.

In some embodiments, the subject is considered to be at risk for exposure to mercury. Risks for mercury exposure include having dental amalgams, which can comprise up to 50% elemental mercury, residing in a close proximity to coal-burning power plants or other sources of mercury pollution, or having a diet with a high mercury content, such as consuming predatory fish, on a semi-regular basis.

In some embodiments of the method, the subject possessing the CPOX4 polymorphism in at least one allele of the CPOX gene has an increased susceptibility to developing a neurobehavioral deficit with exposure to a low level of mercury. As is well-understood in the art, individuals can generally be exposed to three different types of mercury, elemental mercury (“Hg⁰”), inorganic mercury, and organic mercury, each of which has various capacities to enter the body and cause detrimental effects. Elemental mercury is liquid and gives off vapor at room temperature. The vapor can be inhaled and is readily absorbed into the blood and distributed throughout the body, including the brain. Low levels of vapor exposure can include, for example, ranges of mercury vapor concentrations of 0.01 to 50 μg/m³. While Hg⁰ can enter the body through direct skin contact or ingestion, these routes are much less efficient compared to the respiratory route. Inorganic mercury compounds can enter the body through inhalation and ingestion. Organic mercury compounds can enter the body readily through inhalation, ingestion, and dermal routes.

An exposure to low levels of mercury can be defined by the amount of mercury detectable in the body. Mercury in its various forms can be detected in the body according to techniques well-known in the art. For example, blood tests based on spectrometry techniques (e.g., cold vapor atomic fluorescence spectrometry and inductively coupled plasma-mass spectrometry) can be applied to detect all forms of mercury in the body. Exposure to low levels of mercury can be determined, for example, when a blood-based assay indicates approximately 0.5 to approximately 3 micrograms (μg) per 100 milliliters (ml) blood, or any value therein, for example approximately 0.5, 1, 1.5, 2, 2.5, and 3 μg per 100 ml blood. However, blood tests are most effective after acute exposures because the mercury only remains in the blood for a limited number of days after exposure before it passes to tissues. Similarly, Hg⁰ and inorganic mercury can be detected to similar assays performed on urine samples obtained from the subjects. Exposure to low levels of mercury can be determined, for example, when a urine-based assay indicates approximately 0.5 to approximately 30 μg/L mercury. Typically, urinary mercury levels are adjusted for the concentration of a constant component, such as creatinine. Accordingly, the concentration of the mercury can be normalized for concentration of creatinine in the urine sample, and can be expressed as μg mercury per gram (g) creatinine. Low levels of mercury exposure can be defined as exposure resulting in approximately 0.05 to approximately 10 g mercury per gram of creatinine, or any value therein, such as 0.5, 1, 1.5, 2, 2.5, 3, 3.5, 4, 4.5, 5, 6, 6.5, 7, 7.5, 8, 8.5, 9, 9.5, and 10 μg/g creatinine. In some embodiments, low levels of mercury exposure would be determined with detectable urinary mercury levels of 5 μg/g creatinine or less.

In some embodiments of the method, the subject possessing the CPOX4 polymorphism in at least one allele of the CPOX gene can be determined to have an increased susceptibility to developing a neurobehavioral deficit after an acute exposure to mercury. As used herein, the term “acute exposure” refers to a single exposure or continuous or repeated exposure(s) over a short period of time. Exemplary periods for an acute exposure range from a single instance (i.e., over seconds or minutes) to several months, up to two years. For example, as described below, the inventors assayed the impact of acute exposures to mercury at two years after the initiation of the clinical trial.

In some embodiments of the method, the subject possessing the CPOX4 polymorphism in at least one allele of the CPOX gene can be determined to have an increased susceptibility to developing a neurobehavioral deficit after a chronic exposure to mercury. As used herein, the term “chronic exposure” refers to a continuous or substantially continuous exposure, or repeated exposures, to mercury over a prolonged period of time. Exemplary periods for a chronic exposure include periods of more than two years. For example, as described below, the inventors assayed the impact of chronic exposure to mercury over seven years after the initiation of the clinical trial. The chronic exposure was analyzed using the maximum mercury levels and the cumulative mercury levels detected in the subjects over the period of the trial.

In some embodiments of the method, a second assay is performed on a second biological sample obtained from the subject to determine the mercury exposure status of the subject. In some embodiments, the second biological sample comprises urine, blood, serum, plasma, or hair. The second assay can be performed according to techniques known in the art, as described above. For example, as described, assays used to determine mercury exposure include cold vapor atomic fluorescence spectrometry and inductively coupled plasma-mass spectrometry. If the assay is performed on a urine sample, the mercury content is normalized for the concentration of creatinine according to standard techniques. Subjects determined to have both an increased susceptibility to the adverse effects of mercury in addition to measurable mercury exposure can be advised to reduce further exposure to mercury, undergo subsequent monitoring for neurobehavioral effects of exposure, and or undergo ameliorative therapies.

The following is a description of the identification of the CPOX4 allele as a synergistic contributor to the adverse neurobehavioral effects of Hg° exposure in children.

### The Study Population

The study cohort consisted of 330 children (164 boys and 166 girls) for whom CPOX4 or CPOX5 gene status was available from among 507 total subjects enrolled at the start of the clinical trial. Excluded subjects either did not provide a blood sample at the initiation of the study, or were lost to follow-up for acquisition of a buccal cell sample following completion of the trial. Table 1 presents the characteristics of the cohort at Entry as well as at Years 2 and 7 of the clinical trial. Both boys and girls averaged 10.1 years of age, and most were in the 4th grade at entry into the study. Approximately 74% of boys and 71% girls at Entry were Caucasian, and each had an average non-verbal IQ score of 86. Table 1 also displays the mean “Raw” values for urinary IHg concentrations (“HgU”) and the natural log calculations for the HgU at Entry, Year 2 and Year 7 of the clinical trial, as well as the calculated maximum and cumulative values for both sexes at Year 7. The change in number of total subjects between Entry and Year 7 reflects the overall 14% loss to follow-up over the course of the clinical trial (DeRouen, T. A., et al., “Neurobehavioral Effects of Dental Amalgam in Children: a Randomized Clinical Trial,” JAMA 295(15):1784-1792, 2006). The frequencies of the homozygous common (wild type), heterozygous, and homozygous mutant alleles for boys and girls for CPOX4 and CPOX5 are also presented.

TABLE 1
Study population characteristics for participants at Entry (baseline) and in Year 2 and Year 7.

| | BOYS | | | GIRLS | | |
|---|---|---|---|---|---|---|
| | ENTRY | YEAR 2 | YEAR 7 | ENTRY | YEAR 2 | YEAR 7 |
| Characteristic | Mean (SD) | Mean (SD) | Mean (SD) | Mean (SD) | Mean (SD) | Mean (SD) |
| Age | 10.15 (.84) | 12.20 (.84) | 17.14 (.86) | 10.10 (.92) | 12.16 (.95) | 17.08 (1.02) |
| School Year | 4.04 (1.05) | 5.78 (1.2) | 9.40 (2.04) | 4.15 (1.07) | 5.92 (1.16) | 9.86 (1.52) |
| Non-Verbal IQ (at entry only) | 86.26 (10.37) | — | — | 86.26 (10.37) | — | — |
| Urinary Mercury Concentrations | | | | | | |
| Raw HgU^{a} | 1.65 (1.25) | 2.17 (2.02) | 1.25 (3.00) | 1.98 (2.40) | 2.86 (2.63) | 1.77 (2.27) |
| Calculated HgU^{b} | 0.89 (0.41) | 1.02 (0.49) | 0.62 (0.48) | 0.94 (0.48) | 1.18 (0.57) | 0.83 (0.56) |
| Calculated Maximum^{b} | — | — | 1.46 (0.52) | — | — | 1.68 (0.54) |
| Calculated Cumulative^{c} | — | — | 2.47 (0.50) | — | — | 2.74 (0.55) |
| Distribution | % (N) | % (N) | % (N) | % (N) | % (N) | % (N) |
| Total Subjects (N) | 164 | 160 | 121 | 166 | 151 | 118 |
| Caucasian - % (N) | 74.4% (122) | 74.4% (119) | 71.9% (87) | 71.1% (118) | 68.9% (104) | 69.5% (82) |
| CPOX4 | | | | | | |
| Wild type (A/A) | 71.3% (117) | 71.3% (114) | 67.8% (82) | 65.1% (108) | 64.2% (97) | 61.9% (73) |
| Heterozygous (A/C) | 28.0% (46) | 28.1% (45) | 31.4% (38) | 27.7% (46) | 27.8% (42) | 28.8% (34) |
| Homozygous Mutant C/C | 0.6% (1) | 0.6% (1) | 0.8% (1) | 7.2% (12) | 7.9% (12) | 9.3% (11) |
| CPOX5 | | | | | | |
| Wild type (G/G) | 54.3% (89) | 53.8% (86) | 54.2% (65) | 61.7% (103) | 62.5% (95) | 64.7% (77) |
| Heterozygous (G/A) | 37.8% (62) | 38.1% (61) | 38.3% (46) | 32.9% (55) | 32.9% (50) | 31.1% (37) |
| Homozygous Mutant (A/A) | 7.9% (13) | 8.1% (13) | 7.5% (9) | 5.4% (9) | 4.6% (7) | 4.2% (5) |

^{a}μg/g creatinine;
^{b}ln(μg/g creatinine + 1);
^{c}ln[(Σμg/g creatinine) + 1]

### CPOX4 Analyses: Effects on Acute Hg⁰ Exposure

Results of analyses of modification by the CPOX4 variant on neurobehavioral test outcomes associated with acute Hg⁰ exposure (based on HgU at Year 2) are presented in Table 2. Subjects heterozygous and homozygous for the CPOX4 SNP allele are grouped for purposes of these studies. Standard Error (SE) and p values (p) are indicated in parentheses. Among boys, significant interaction effects between CPOX4 and Hg⁰ exposure were observed for three of the 23 tests of neurobehavioral function evaluated during the clinical trial. These included the Stroop color and word/color tests, both within the Attention domain, and dominant hand finger tapping, a test of motor function within the Motor domain. The significant dose-response relationships between performance and acute Hg⁰ exposure for these tests were all in the impaired (adverse) direction and were restricted to boys with at least one CPOX4 variant allele. There were no main effects observed for any of the other tests.

Among girls, CPOX4 significantly modified the effects of acute Hg⁰ exposure on two tests of neurobehavioral function, the RAVALT Trial 5 and RAVALT Trial 8-List 20′ tests, both within the Learning and Memory domain (Table 2). In both cases, significantly decreased performance associated with acute Hg⁰ exposure was observed only among girls with at least one CPOX4 variant allele. Also among girls, 4 tests were observed to have significant main effects associated with acute Hg⁰ exposure (not shown). Among these, only the Trails A test (p≦0.03) was in the impaired direction, while 3 tests, the Stroop Color/Word test (p≦0.03), the RAVALT Trial 6 test (p≦0.02), and the Peg test for the dominant hand (p≦0.03), had significant associations in the improved direction. In addition, two tests, finger tapping dominant (p≦0.001) and non-dominant (p≦0.01), had significantly improved performance among girls identified as having at least one CPOX4 variant. Aside from Motor function, no domain of neurobehavioral function was found to have more than one test that achieved statistical significance in terms of either main effect of acute Hg⁰ exposure or CPOX4 gene status among girls.

TABLE 2
Acute Hg⁰ dose-response effects among boys and girls with
CPOX WT or CPOX4 variant in Year 2

| | WT | | Het or Mut | |
|---|---|---|---|---|
| Measure | Beta (SE) | rₚₐᵣₜ (p) | Beta (SE) | rₚₐᵣₜ (P) |

BOYS
Attention Domain

| | | | | |
|---|---|---|---|---|
| Stroop Test - | −.19 (1.80) | −.01 (.92) | −8.40 (2.80)* | −.42 (.005)* |
| Color | | | | |
| Stroop Test - | 1.09 (1.51) | .07 (.47) | −3.50 (1.68)* | −.31 (.04)* |
| Word/Color | | | | |

Motor Domain

| | | | | |
|---|---|---|---|---|
| Finger | 1.21 (.91) | .13 (.19) | −3.25 (1.53)* | −.32 (.04)* |
| Tapping - | | | | |
| Dominant | | | | |

GIRLS
Learning and Memory Domain

| | | | | |
|---|---|---|---|---|
| RAVALT | .28 (.43) | .07 (.51) | −1.34 (.47)* | −.38 (.006)* |
| TR 5 | | | | |
| RAVALT | .34 (.45) | .08 (.46) | −1.69 (.55)* | −.40 (.003)* |
| TR 8 - | | | | |
| List A 20′ | | | | |

Values marked with an asterisk (*) signify p ≦ 0.05.

### CPOX4 Analyses: Effects on Chronic Hg⁰ Exposure Among Boys

Analysis of the effects of CPOX4 on neurobehavioral performance associated with chronic Hg⁰ exposure among boys showed numerous significant interaction terms across a wide range of tests and performance domains (e.g., Attention, Visual-Spatial, Executive Function, Learning & Memory, and Motor). These results are presented in Table 3. When cumulative exposure was used as the chronic Hg⁰ exposure measure, significant Hg⁰ dose-response effects were observed on 11 of 23 neurobehavioral test outcomes among boys with at least one CPOX4 variant allele, all in the impaired (reduced) direction, with 7 associations being significant at p≦0.01. In contrast, no significant cumulative Hg⁰ dose-response effects on neurobehavioral test performance were observed among boys genotyped as CPOX wild type.

Using maximum exposure as the chronic Hg⁰ exposure matrix, 14 tests demonstrated significant dose-response relationships among boys with CPOX4 variant status, 12 being significant at p≦0.01. The 15th test (WRAVMA Pegs Non-Dominant) reached near significance (p≦0.06). All of these effects are in the impaired (reduced) direction in that performance is significantly adversely affected in relation to Hg⁰ exposure among boys with CPOX4 variant status. In contrast, among boys genotyped as CPOX wild type, only one neurobehavioral function, Finger tapping-Dominant, was significantly associated with maximum Hg⁰ exposure (p≦0.03), and this was in the improved direction (Table 4).

TABLE 3
Chronic Hg⁰ dose-responsive effects among boys with CPOX WT or CPOX4 variant alleles in Year 7.

| | | Cumulative Hg⁰ | | | | Maximum Hg⁰ | | | |
|---|---|---|---|---|---|---|---|---|---|
| Behavioral | | WT | | HET OR MUT | | WT | | HET OR MUT | |
| Test | N | Beta (SE) | ^{r}part^{(p)} | Beta (SE) | ^{r}part^{(p)} | Beta (SE) | ^{r}part^{(p)} | Beta (SE) | ^{r}part^{(p)} |

ATTENTION

| | | | | | | | | | |
|---|---|---|---|---|---|---|---|---|---|
| Stroop Test - | 121 | −1.14 (2.32) | −.06 (.63) | −12.55 (4.05)* | −.47 (.004)* | .73 (2.24) | .04 (.74) | −11.17 (3.80) | −.45 (.006) |
| Color | | | | | | | | | |
| Word | 121 | −2.90 (3.20) | −.10 (.37) | −18.06 (4.89)* | −.54 (.001)* | −.77 (3.10) | −.03 (.80) | −17.10 (4.51) | −.54 (.001) |
| WAIS-III - | 121 | .11 (.77) | .02 (.88) | −3.57 (1.12)* | −.48 (.003)* | .87 (.74) | .13 (.24) | −3.90 (.98) | −.57 (.0001) |
| Digit Span | | | | | | | | | |
| WMS-III - | 121 | .17 (.62) | .03 (.79) | −2.45 (.97)* | −.40 (.02)* | .57 (.60) | .11 (.35) | −2.89 (.85) | −.50 (.002) |
| Spatial Span | | | | | | | | | |
| Adult Trails | 120 | −.80 (2.07) | −.04 (.70) | 11.25 (3.83)* | .45 (.006)* | −2.37 (1.98) | −.14 (.24) | 13.04 (3.30) | .56 (.0001) |
| A - Time | | | | | | | | | |
| (Sec) | | | | | | | | | |

VISUAL-SPATIAL

| | | | | | | | | | |
|---|---|---|---|---|---|---|---|---|---|
| SRT Mean | 121 | .00 (.03) | .01 (.96) | .15 (.05)* | .44 (.007)* | .01 (.03) | .02 (.86) | .14 (.05)* | .46 (.005)* |
| WAIS-III - | 120 | −2.46 (3.33) | −.08 (.46) | −20.70 (6.08)* | −.50 (.002)* | −.32 (3.32) | −.01 (.92) | −18.17 (5.75)* | −.48 (.003)* |
| Digit | | | | | | | | | |
| Symbol | | | | | | | | | |

EXECUTIVE FUNCTION

| | | | | | | | | | |
|---|---|---|---|---|---|---|---|---|---|
| Adult Trails | 120 | — | — | — | — | 1.98 (6.08) | .04 (.75) | 22.26 (7.03)* | .48 (.003)* |
| B | | | | | | | | | |

LEARNING & MEMORY

| | | | | | | | | | |
|---|---|---|---|---|---|---|---|---|---|
| RAVALT | 121 | — | — | — | — | .51 (.43) | .13 (.24) | −1.78 (.80)* | −.36 (.03)* |
| TR5 - List A | | | | | | | | | |
| TR7 - A/ | 120 | — | — | — | — | .52 (.52) | .11 (.33) | −2.24 (.82)* | −.42 (.01)* |
| post B | | | | | | | | | |
| TR8 - List | 120 | .32 (.60) | .06 (.60) | −2.01 (.92)* | −.35 (.04)* | 84 (.58) | .16 (.15) | −2.14 (.83)* | −.40 (.01)* |
| A 20′ | | | | | | | | | |

MOTOR

| | | | | | | | | | |
|---|---|---|---|---|---|---|---|---|---|
| WRAVMA - | 121 | — | — | — | — | −.55 (1.58) | −.04 (.73) | −7.16 (2.90)* | −.39 (.02)* |
| Pegs | | | | | | | | | |
| Dominant | | | | | | | | | |
| Non Dom | 121 | .41 (1.49) | .03 (.27) | −5.87 (2.92)* | −.33 (.05)* | .79 (1.44) | .06 (.58) | −5.34 (2.72) | −.32 (.06) |
| Finger | 119 | 1.59 (1.22) | .15 (.20) | −4.03 (1.76)* | −.37 (.03)* | 2.59 | .25 | −3.46 (1.65)* | −.34 (.04)* |
| Tapping - | | | | | | (1.15)# | (.03)# | | |
| Dominant | | | | | | | | | |
| Non Dom | 119 | 1.07 (1.27) | .10 (.40) | −7.24 (1.67)* | −.60 (.0001)* | 2.14 (1.21) | 20 (.08) | − 5.83 (1.66)* | −.52 (.001*) |

Values marked with an asterisk (*) signify p ≦ 0.05 in the impaired (reduced) direction.
Values marked with a pound (#) signify p ≦ 0.05 in the improved direction.

Table 4 presents the significant main effect relationships for tests of neurobehavioral performance for which significant interaction terms were not observed among boys in the present analyses. When evaluated in terms of the cumulative chronic Hg⁰ exposure matrix, four tests of neurobehavioral performance were significantly associated with Hg⁰ exposure and one test was specifically associated with CPOX4 gene status. While all of these associations were in the impaired (reduced) direction, most were of only borderline significance in terms of suggesting independent effects of either chronic Hg⁰ exposure or CPOX4 gene status on neurobehavioral performance. When evaluated in terms of the maximum chronic Hg⁰ exposure matrix, three test outcomes were found to be significantly associated with Hg⁰ exposure, but none specifically with the CPOX4 variant.

TABLE 4
Significant main effects for chronic Hg⁰ exposure and CPOX4 among boys in Year 7.

| | Cumulative Hg | | | | Maximum Hg | | | |
|---|---|---|---|---|---|---|---|---|
| Behavioral | Hg (ln) | | CPOX4 | | Hg (ln) | | CPOX4 | |
| Test | Beta (SE) | ^{r}part^{(p)} | Beta (SE) | ^{r}part^{(p)} | Beta (SE) | ^{r}part^{(p)} | Beta (SE) | ^{r}part^{(p)} |

ATTENTION

| | | | | | | | | |
|---|---|---|---|---|---|---|---|---|
| Stroop Test - Word/Color | −3.30 (1.64)* | −.18 (.05)* | −1.50 (1.78) | −.08 (.40) | −2.17 (.59) | −.13 (.16) | −1.64 (1.79) | −.08 (.36) |

VISUAL-SPATIAL

| | | | | | | | | |
|---|---|---|---|---|---|---|---|---|
| WAIS-III - Symbol Search | −3.48 (1.54)* | −.20 (.03)* | 1.86 (1.67) | .10 (.27) | −2.91 (1.49)* | −.18 (.05)* | 1.73 (1.68) | .10 (.30) |

LEARNING & MEMORY

| | | | | | | | | |
|---|---|---|---|---|---|---|---|---|
| WMS-III - Visual | −1.66 (.83)* | −.18 (.05)* | −.06 (.90) | −.01 (.95) | −1.63 (.80)* | −.19 (.04)* | −.11 (.90) | −.01 (.90) |
| Reproduction - Immediate | | | | | | | | |
| Delayed | −2.95 (1.24)* | −.22 (.02)* | .62 (1.34) | .04 (.64) | −2.70 (1.19)* | −.21 (.02)* | .52 (1.34) | .04 (.70) |

MOTOR

| | | | | | | | | |
|---|---|---|---|---|---|---|---|---|
| WRAVMA - Pegs Dominant | .41 (1.49) | .03 (.27) | −5.87 (2.92)* | −.33 (.05)* | — | — | — | — |

Values marked with an asterisk (*) signify p ≦ 0.05.

### CPOX4 Analyses: Effects on Chronic Hg⁰ Exposure Among Girls

In contrast to findings among boys, no significant dose-response relationships for Hg⁰ exposure were observed for either maximum or cumulative measures of chronic Hg⁰ exposure when evaluated separately among CPOX wild type girls or girls with at least one CPOX4 variant allele, despite the fact that two tests (i.e., WAIS-III Digit Span test and RAVALT Trial 6—List B test) had significant interaction terms between CPOX4 and maximum chronic exposure. Main effects analyses among all girls found no significant associations with either measure of chronic Hg⁰ exposure. However, two tests (i.e., finger tapping—dominant test and simple reaction time test) had significant associations (p≦0.01 or p≦0.02, respectively) with CPOX4 for both chronic Hg⁰ measures. Three of these four associations showed significantly improved performance among girls identified as having at least one CPOX4 variant versus those with CPOX wild type.

### Analyses of CPOX4 Effects by Neurobehavioral Domain

1. Attention

The Attention domain includes 3 Stroop subtests, the Digit and Spatial Span tests (considered to be more related to Attention and rehearsal than to Memory), and the Trails A test. One of the Stroop tests, the Color/Word test, introduces discordance between the color spelled out and the color in which the word is written. This test requires what may be called “directed attention,” in that it includes areas of brain function other than those specifically associated with attention, and thus is not as direct a measure of simple attention as the other tests.

Among the CPOX4 acute effects analyses (Table 2), two of the Stroop subtests (Color and Color/Word) had significant dose-response associations among boys with the CPOX4 gene variant. The CPOX4 chronic effects analyses (Table 3) involved all of the Attention Domain tests except for the Stroop Color/Word sub-test. These analyses suggest that Attention is highly impacted by Hg⁰ exposure among boys with CPOX4 variant status when evaluated in terms of either maximum or cumulative Hg⁰ exposure.

2. Visual-Spatial Acuity

The Visual-Spatial domain tests include Simple Reaction Time (responding to a visual stimulus), Digit Symbol (coding a symbol with a digit), and Symbol Search (scanning to find target symbols). As shown in Table 3, significant associations for tests of Visual-Spatial acuity were found for analyses employing measures of chronic but not acute Hg⁰ exposure among boys. Two tests, Simple Reaction Time and Digit Symbol, had significant dose-response associations with both maximum and cumulative chronic exposure matrices among boys genotyped as having at least one CPOX4 variant allele, but not boys with CPOX wild type status. Additionally, in the main effects analyses (Table 4), the Symbol Search test had significant associations with both chronic Hg⁰ exposure matrices. These analyses show that the Visual-Spatial domain can be impacted by Hg⁰ exposures within this genetic subgroup.

3. Executive Function

Executive Function tests included Wisconsin Card Sort (sorting cards by changing criteria) and Adult Trails B (following a trail of alternating numbers and alphabet letters). As shown in Table 3, the presence of CPOX4 was found to significantly modify the effect of Hg⁰ exposure on only the latter test of Executive Function among boys, and this effect was restricted to the analysis employing the maximum exposure matrix as a measure of chronic Hg⁰ exposure. This effect, however, was in the impaired (reduced) direction and the association was highly significant (p≦0.003).

4. Learning & Memory

The Learning & Memory domain included nine measures consisting of five sub-tests of the RAVALT (an auditory verbal learning test using a list of 15 words with distraction and delayed recall), two Visual Reproductions tests (redrawing a figure immediately and delayed), and the CVMT test (recalling which visual objects are repeated). Among the analyses assessing the effects of CPOX4 variant status on acute Hg⁰ exposure effects (Table 2), CPOX4 variant status was found to significantly modify the adverse effects of acute Hg⁰ exposure on two of the RAVALT sub-tests (Trial 5 and Trial 8—list A 20′) among girls, constituting the principal positive observation regarding the interaction of CPOX4 and Hg⁰ exposure among girls in this study. No associations of gene status with indices of chronic Hg⁰ exposure among girls were observed.

In contrast, three RAVALT sub-tests were found to have significant dose-response associations with the maximum Hg⁰ chronic exposure matrix among boys with the CPOX4 variant (Table 3), and one also having a significant association with the cumulative Hg⁰ exposure matrix. In the main effects analyses (Table 4), both immediate and delayed Visual Reproductions tests were found to have significant independent associations with both chronic Hg⁰ exposure matrices.

5. Motor Function

The Motor function domain tests include Finger Tapping (number of taps in a fixed time) and the Pegs test (number of dowels inserted into a hole in a fixed time). Both tests evaluate dominant and non-dominant hand performance separately. All four of these tests either approached significance or were significantly associated with the maximum exposure matrix among boys carrying the CPOX4 variant, with one association in the improved direction among boys genotyped as CPOX wild type (Table 3). There were three tests (excluding Peg dominant hand) related to the cumulative Hg⁰ exposure matrix among boys with the CPOX4 variant. Whereas most of the associations observed in this domain demonstrated only moderate significance levels, the interaction of CPOX4 and Hg⁰ exposure on the Finger Tapping non-dominant hand test was highly significant.

5. CPOX5 Analyses

No significant interaction effects were observed for CPOX5 and any measure of Hg⁰ exposure among either boys or girls. Moreover, no main effects were observed for CPOX5.

6. Summary of Results

CPOX4 gene status was found to modify the adverse effects of chronic Hg⁰ exposure on a wide range of neurobehavioral performance test results among boys. All of these effects were in the impaired (reduced) direction and spanned all five neurobehavioral domains. Many of the observed associations were highly significant (p≦0.01). These highly consistent associations, affecting more than half the neurobehavioral tests evaluated, cannot be dismissed, due to the large numbers of tests and exposures evaluated. The converse lack of associations between Hg⁰ exposure and CPOX wild type status suggests that the CPOX4 variant may be important in mediating neurotoxic effects of Hg⁰ exposure in boys. The absence of associations when comparable analyses were conducted for the CPOX5 variant argues against these observations being due to some selection process.

### Discussion

Numerous studies have proposed a component of genetic susceptibility to neurobehavioral disorders associated with mercury and other xenobiotic exposures (Braun, J. M., et al., “Exposures to Environmental Toxicants and Attention Deficit Hyperactivity Disorder in U.S. Children,” Environ. Health Perspect. 114:1904-1909, 2006; Gundacker, C., et al., “The Relevance of Individual Genetic Background for the Toxicokinetics of Two Significant Neurodevelopmental Toxicants: Mercury and Lead,” Mut. Res. 705:130-140, 2010; Engström, K. S., et al., “Genetic Variation in Glutathione-Related Genes and Body Burden of Methylmercury,” Environ. Health Perspect. 116:734-739, 2008; Suk, W. A., and G. W. Collman, “Genes and the Environment: Their Impact on Children's Health,” Environ. Health Perspect. 106(3):817-820, 1998), although the modifying effects of commonly expressed genetic variants on these associations are just beginning to be defined. The present study is believed to be the first to describe a genetic polymorphism that modifies the effects of elemental mercury exposure on a wide variety of neurobehavioral functions in children. Previous studies provided evidence of significant associations between Hg⁰ exposure and the CPOX4 variant on neurobehavioral functions in adult dental professionals (Echeverria, D., et al., “The Association Between a Genetic Polymorphism of Coproporphyrinogen Oxidase, Dental Mercury Exposure, and Neurobehavioral Response in Humans,” Neurotox. Teratol. 28:39-48, 2006), although the observed joint effects in that study were found to be strictly additive in nature. In contrast, the present study surprisingly demonstrates synergistic, i.e., more than additive, interactions between Hg⁰ and CPOX4 on numerous neurobehavioral functions in children. These results indicate a heightened susceptibility of children to the adverse neurobehavioral effects of Hg⁰ that are specifically associated with the presence of the CPOX4 genetic variant.

The paucity of independent effects of Hg⁰ exposure on tests of neurobehavioral function in this study provide some consistency with findings from the dental amalgam clinical trial (DeRouen, T. A., et al., “Neurobehavioral Effects of Dental Amalgam in Children: a Randomized Clinical Trial,” JAMA 295(15):1784-1792, 2006), in which exposure to Hg⁰ from dental amalgam was found not to be associated with deficits in any tests of neurobehavioral performance among either boys or girls. However, when controlling for CPOX gene status as performed here, Hg⁰ exposure was strongly associated with diminished performance across a wide range of the same tests, especially among boys with the CPOX4 variant. Diminished performance was most predominantly observed in tests of Attention, suggesting possible impairment of attentional vitality and flexibility, e.g., ability to sustain attention or to shift between two sequences held in working memory (Echeverria, D., et al., “Test-Retest Reliability and Factor Stability of the Behavioral Evaluation for Epidemiology Studies Test Battery,” Percept. Motor Skills 95:845-867, 2002). Significant interactions between Hg⁰ exposure and CPOX4 on tests of Learning & Memory and of Visual-Spatial acuity were also observed, suggesting possible decrements of verbal learning and memory as well as of perceptual cognition.

Effects on tests of Motor function, including measures of manual coordination and fine motor speed, also appear to be adversely affected when evaluated within the context of chronic Hg⁰ exposure among boys with the CPOX4 variant. These findings have important public health implications, inasmuch as mean urinary Hg levels among boys in this study ranged from 1.4 (1.3-1.6) μg/g creatinine at baseline to a maximum of 2.2 (1.8-2.5) μg/g creatinine at Year 2 of follow-up in the dental amalgam clinical trial. By comparison, geometric mean urinary mercury levels measured among a nationally representative sample of children 12-19 years of age acquired as part of the 2003-2004 U.S. National Health and Nutrition Examination Survey (National Health and Nutrition Examination Survey, Centers for Disease Control and Prevention, 2007, available at the world wide web domain cdc.gov/nchs/nhanes.htm [as of Feb. 6, 2012]) were 0.358 (0.313-0.408) μg/g creatinine. Although this value is substantially lower than those measured in the present study, the mean urinary Hg concentration in the 90th percentile of that sample was 1.59 (1.13-2.52) μg/g creatinine, which is comparable to the range of Hg concentrations at which adverse neurologic effects of Hg⁰ were observed herein among boys with CPOX4. These observations suggest potential adverse neurobehavioral effects of Hg⁰ among boys with the CPOX4 variant who fall within the top 10% of subjects sampled within that survey for Hg⁰ exposure.

The mechanistic association of CPOX4 to neurobehavioral functions remains to be delineated. Without being bound to any particular theory, potential alterations in physiological heme availability and/or heme-dependent processes associated with diminished CPOX4 activity may underlie this effect (Li, T., and J. S. Woods, “Cloning, Expression, and Biochemical Properties of CPOX4, a Genetic Variant of Coproporphyrinogen Oxidase That Affects Susceptibility to Mercury Toxicity in Humans,” Toxicol. Sci. 109:228-236, 2009). In this regard, heme is known to play a critical role as a signaling molecule in glutaminergic neuronal receptor processing and synapse development (Chemova, T., et al. “Heme Deficiency Is Associated With Senescence and Causes Suppression of N-Methyl-D-Aspartate Receptor Subunits Expression in Primary Cortical Neurons,” Molec. Pharmacol. 69:697-705, 2006; Sengupta, A., et al., “Heme Deficiency Suppresses the Expression of Key Neuronal Genes and Causes Neuronal Cell Death,” Brain Res. Molec. Brain Res. 137:23-30, 2005), as well as in the regulation of serotonin (5-hydroxytryptamine) synthesis and signaling in the central nervous system (Litman, D. A., and M. A. Correia, “L-Tryptophan: A Common Denominator of Biochemical and Neurological Events of Acute Hepatic Porphyria?” Science 222:1031-1033, 1983; Litman, D. A., and M. A. Correia, “Elevated Brain Tryptophan and Enhanced 5-Hydroxytryptamine Turnover in Acute Hepatic Heme Deficiency: Clinical Implications,” J. Pharmacol. Exper. Therap. 232:337-345, 1985). Disorders of both systems have been implicated as etiologic in a variety of neurodevelopmental and neurobehavioral disorders (Chemova, T., et al., “Early Failure of N-Methyl-D-Aspartate Receptors and Deficient Spine Formation Induced by Reduction of Regulatory Heme in Neurons,” Molec. Pharmacol. 79:844-854, 2011; Chugani, D. C., et al. “Developmental Changes in Brain Serotonin Synthesis Capacity in Autistic and Nonautistic Children,” Ann. Neurol. 45:287-295, 1999), and both could be amenable to disruption by heme deficiency during critical periods of neurological development in children, particularly in association with mercury exposure (Li, T., and J. S. Woods, “Cloning, Expression, and Biochemical Properties of CPOX4, a Genetic Variant of Coproporphyrinogen Oxidase That Affects Susceptibility to Mercury Toxicity in Humans,” Toxicol. Sci. 109:228-236, 2009). While these observations provide a scientific rationale for the diminished neurobehavioral performance observed here among boys with the CPOX4 variant and Hg⁰ exposure, further studies are required to define the specific mechanistic events underlying this association.

The absence of effects of CPOX5 on neurobehavioral functions when evaluated in relation to any measure of Hg⁰ exposure in this study suggests that the CPOX4 variant may act in a genotype-selective manner to mediate the adverse neurobehavioral effects of Hg⁰ exposure observed here. While the potential effects of CPOX5 on CPOX enzymatic activity, heme bioavailability, or processes affecting neurological function are not known, CPOX5 need not be viewed as incapable of affecting biological processes, inasmuch as synonymous SNPs are widely recognized as mediating changes in translation kinetics, protein folding and other factors that underlie a wide variety of neurological and other disorders in humans (Chamary, J. V., et al., “Hearing Silence: Non-Neutral Evolution at Synonymous Sites in Mammals,” Nat. Rev. Genet. 7:98-108, 2006; Duan, J., et al., “Synonymous Mutations in the Human Dopamine Receptor D2 (DRD2) Affect mRNA Stability and Synthesis of the Receptor,” Hum. Molec. Genet. 112:205-216, 2003; Komar, A. A., “SNPs, Silent but Not Invisible,” Science 315:466-467, 2007). Moreover, the heterozygous and full mutant variants of CPOX5 were distributed quite differently from those of CPOX4 within this cohort. Only 7 subjects (2%) shared both CPOX4 and CPOX5 variant status, militating against selection bias in terms of findings observed with respect to those with CPOX4. Further research analyzing multiple SNPs within the CPOX gene as well as others associated with heme-dependent neurotransmitter processing pathways is required to identify the mechanisms underlying the apparent selective effects of CPOX4 seen here.

Notable differences between boys and girls in the effects of Hg⁰ exposure and the CPOX4 variant on neurobehavioral test performance were observed in this study. Although Hg⁰ exposure from dental amalgam was comparable among boys and girls participating in the clinical trial (DeRouen, T. A., et al., “Neurobehavioral Effects of Dental Amalgam in Children: a Randomized Clinical Trial,” JAMA 295(15):1784-1792, 2006), sex-related differences in Hg toxicokinetics that may afford greater Hg excretion and, consequently, lesser likelihood of Hg retention and accumulation in girls than boys may contribute to this effect (Woods, J. S., et al., “The Contribution of Dental Amalgam to Urinary Mercury Excretion in Children,” Environ. Health Perspect. 115:1527-1531, 2007). Numerous other factors that include genetic and hormonal differences affecting brain development, structure and function between boys and girls are also likely to contribute to the gender differences observed here (Gochfield, M., “Framework for Gender Differences in Human and Animal Toxicology,” Environ. Res. 104:4-21, 2007; Hines, R. N., et al., “Approaches for Assessing Risk to Sensitive Populations: Lessons Learned From Evaluating Risk in the Pediatric Population,” Toxicol. Sci. 113:4-26, 2010; Vahter, M., et al., “Genetic Differences in the Disposition and Toxicity of Metals,” Environ. Res. 104:85-95, 2007; Valentino, R. J., “Molecular and Cellular Sex Differences at the Intersection of Stress and Arousal,” Neuropharmacol. 62:13-20, 2012). Differences in detection sensitivity for CPOX4 between boys and girls in this study have a less clear explanation, although genetic factors underlying gender differences in numerous psychiatric and neurobehavioral disorders have been reported (Baca-Garda, E., et al., “A Gender-Specific Association Between the Serotonin Transporter Gene and Suicide Attempts,” Neuropsychopharmacol. 26:692-695, 2002; Gaub, M., and C. L. Carlson, “Gender Differences in ADHD: a Meta-Analysis and Critical Review,” J. Amer. Acad. Child Adoles. Psychiat. 36:1036-1045, 1997; Harrison, P. J., and E. M. Tunbridge, “Catechol-O-Methyltransferase (COMT): A Gene Contributing to Sex Differences in Brain Function, and to Sexual Dimorphism in the Predisposition to Psychiatric Disorders,” Neuropsychopharmacol 33:3037-3045, 2008; Samochowiec, J., et al. “Association Studies of MAO-A, COMT, and 5-HTT Genes Polymorphisms in Patients With Anxiety Disorders of the Phobic Spectrum,” Psychiat. Res. 128(1):21-26, 2004). The observation that neither Hg⁰ exposure nor CPOX4 alone substantially affected neurobehavioral performance in girls suggests that sex-related predisposition, in addition to differences in Hg toxicokinetics, affects susceptibility. Of note, measures of cognitive function and other behaviors not specifically related to reproduction are often sex-linked, accounting for substantial differences in response to many chemical agents, with subsequent expression in behavior (Weiss, B., “Sexually Dimorphic Nonreproductive Behaviors as Indicators of Endocrine Disruption,” Environ. Health Perspect. 110(suppl. 3):387-391, 2002). The sexually divergent responses to Hg⁰ exposure and genetic disposition observed in the present study highlight the importance of considering such differences in development of strategies aimed at risk assessment and prevention, especially in children.

In conclusion, the present studies demonstrate significant adverse effects on neurobehavioral functions associated with chronic Hg⁰ exposure and the CPOX4 genetic variant among children, with effects manifested predominantly among boys. These findings are the first to describe a genetic polymorphism that modifies the effects of Hg⁰ exposure on neurobehavioral functions in children, and suggest directions for future research to define mechanisms underlying differential sensitivity to mercury between boys and girls.

### EXAMPLES

### Example 1

### Materials and Methods

### The Study Population

The current study was performed on a subset of 330 subjects who participated as children in the Casa Pia Dental Amalgam Clinical Trial (DeRouen, T. A., et al., “Issues in the Design and Analysis of a Randomized Clinical Trial to Assess the Safety of Dental Amalgam Fillings in Children,” Contr. Clin. Trials 23:301-320, 2002; DeRouen, T. A., et al., “Neurobehavioral Effects of Dental Amalgam in Children: a Randomized Clinical Trial,” JAMA 295(15):1784-1792, 2006) conducted between 1996 and 2006. Participants in the clinical trial included 279 boys and 228 girls, aged 8-12 yrs at baseline, who were students of the Casa Pia school system in Lisbon, Portugal. Children were initially randomized to Hg amalgam (treatment) or composite resin (control) dental treatment groups. Subjects were evaluated at baseline and at seven subsequent annual intervals following initial dental treatment using an extensive battery of neurobehavioral assessments (Slade, P. D., et al., “The Serial Use of Child Neurocognitive Tests: Development Versus Practice Effects,” Psychol. Assess. 20:361-369, 2008; Townes, B. D., et al., “A Longitudinal Factor Analytic Study of Children's Neurocognitive Abilities,” Int. J. Neurosci. 118(7):1009-1023, 2008a; Townes, B. D., et al., “Repeat Test Scores on Neurobehavioral Measures Over an Eight-Year Period in a Sample of Portuguese Children,” Int. J. Neurosci. 118(1):79-93, 2008b). Follow-up data were obtained on a similar number of subjects in each treatment group. Studies conducted during the course of the clinical trial (Evens, C. C., et al., “Examination of Dietary Methylmercury Exposure in the Casa Pia Study of the Health Effects of Dental Amalgams in Children,” J. Toxicol. Environ. Health 64:521-530, 2001) demonstrated that the children had no significant exposure to methylmercury from dietary fish consumption.

### Neurobehavioral Tests Employed

A comprehensive neurobehavioral test battery was used in this analysis, including measures from the Rays Verbal Learning Test (RAVALT), subtests from the Wide Range Assessment of Memory and Learning and Visual Motor Abilities (WRAVMA), the Wechsler Intelligence Scale for Children III (WISC-III), and the Wechsler Intelligence Scale for Adults-III (WMS-III), Simple Reaction Time, Finger tapping, Trailmaking A and B, the Stroop test, and Wisconsin Card Sort. The validity and rationale underlying the use of these tests in the clinical trial have been described (Martins, I. P., et al. “Age and Sex Difference in Neurobehavioral Performance: a Study of Portuguese Elementary School Children,” Int. J. Neurosci. 115:1687-1709, 2005).

Table 5 lists the 23 neurobehavioral tests that were assessed and presents their means and standard deviations (SD) at their last year of administration (Year 7). Tests are organized within the behavioral domains that were evaluated in the clinical trial (DeRouen, T. A., et al., “Neurobehavioral Effects of Dental Amalgam in Children: a Randomized Clinical Trial,” JAMA 295(15):1784-1792, 2006). Arrows depict whether the test score increases or decreases in magnitude with improved performance. Impaired performance associated with Hg⁰ exposure or CPOX4 variant status is described as occurring in the “impaired” or “reduced” direction, whereas improved performance associated with either of these variables is described as occurring in the “improved” direction. The Comprehensive Test Of Nonverbal Intelligence (CTONI) was given to each child at the beginning of the clinical trial to obtain a measure of IQ at baseline.

TABLE 5
Neurobehavioral tests assessed with mean scores for Year 7 (Final year of clinical trial).\

| | | | |
|---|---|---|---|
| | | Boys N = 121 | Girls N = 118 |
| Test/Domain | Measure^{a} | Mean (SD) | Mean (SD) |

ATTENTION

| | | | | |
|---|---|---|---|---|
| Stroop Test | Color | # correct ↑ | 66.41 (11.96) | 69.23 (10.43) |
| | Word | # correct ↑ | 90.07 (15.18) | 91.47 (15.23) |
| | Color-Word | # correct ↑ | 41.84 (9.78) | 43.88 (8.75) |
| WAIS-III | Digit Span | # correct ↑ | 14.34 (3.68) | 14.14 (2.77) |
| WMS III | Spatial Span | # correct ↑ | 15.84 (3.02) | 15.58 (3.12) |
| Adult Trails A | | Time (sec) ↓ | 26.35 (10.62) | 30.38 (11.38) |

VISUAL-SPATIAL

| Simple Reaction Time | | Time (sec) ↓ | 0.74 (0.15) | 0.77 (0.13) |
|---|---|---|---|---|
| WAIS III | Digit Symbol | # correct ↑ | 72.45 (17.09) | 76.68 (13.87) |
| | Symbol Search | # correct ↑ | 33.15 (8.85) | 34.57 (8.04) |

EXECUTIVE FUNCTIONING

| | | | |
|---|---|---|---|
| Wisconsin Card Sort - Categories | # categories ↑ | 3.06 (1.38) | 3.08 (1.47) |
| Completed | | | |
| Adult Trails B | Time (sec) ↓ | 65.69 (26.99) | 63.41 (23.67) |

LEARNING & MEMORY

| | | | | |
|---|---|---|---|---|
| RAVALT | Tr1 - List A | # correct ↑ | 5.61 (1.49) | 6.10 (1.86) |
| | Tr5 - List A | # correct ↑ | 11.24 (2.20) | 11.54 (2.23) |
| | Tr6 - List B | # correct ↑ | 4.73 (1.38) | 5.27 (1.56) |
| | Tr7 - List A/Post B | # correct ↑ | 9.85 (2.56) | 10.22 (2.48) |
| | Tr8 - List A after 20′ | # correct ↑ | 9.30 (2.72) | 10.07 (2.77) |
| WMS-III | Visual Reproductions | | | |
| | Immediate | # correct ↑ | 34.72 (4.65) | 36.61 (2.98) |
| | Delayed | # correct ↑ | 31.98 (6.96) | 34.90 (4.02) |
| CVMT d-Prime | | Score ↑ | 1.51 (.94) | 1.61 (.88) |

MOTOR

| | | | | |
|---|---|---|---|---|
| WRAVMA - Pegs | Dominant | # Pegs ↑ | 47.40 (8.50) | 49.92 (6.30) |
| | Non Dominant | # Pegs ↑ | 44.33 (7.57) | 45.02 (6.12) |
| Finger Tapping | Dominant | # Taps ↑ | 52.75 (5.59) | 48.44 (5.72) |
| | Non Dominant | # Taps ↑ | 46.66 (5.90) | 42.53 (5.81) |

^{a}Arrows show direction of improved performance.

### Genotyping Assays

Genotyping for the present study was performed on DNA extracted from buccal cell samples that were obtained from study subjects following completion of the clinical trial (n=199) or from blood samples that were obtained at baseline for blood lead assessments (n=152). Genotyping was performed by the Functional Genomics Laboratory of the NIEHS Center for Ecogenetics and Environmental Health at the University of Washington, using automated DNA sequencing assays. Oligonucleotides used for PCR and sequencing as well as primer and allele-specific probes used for fluorescent 5′-nuclease assays have been previously described in detail (Woods, J. S., et al., “The Association Between Genetic Polymorphisms of Coproporphyrinogen Oxidase and an Atypical Porphyrinogenic Response to Mercury Exposure in Humans,” Toxicol. Appl. Pharmacol. 206:113-120, 2005). In the present study, each child was evaluated for CPOX4 gene status, and categorized as CPOX wild type (A/A) or CPOX4 (A/C or C/C) if either a single or double allelic variant, respectively, was found. Children were also characterized for the CPOX5 variant as CPOX wild type (G/G) or CPOX5 (G/A or A/A) if either a single or double allelic variant, respectively, was found. For CPOX5, see SNP reference identifier rs1729995, where the SNP position is presented at nucleotide position 27 in the disclosed sequence. The sequence is also set forth herein as SEQ ID NO:8, wherein the SNP location at nucleotide position 27 is represented with an N, which can be an A or G.

### Human Subject Considerations

All parents or guardians of children who participated in the clinical trial gave written consent, and all children provided signed assent, for the treatments and assessments made during the course of the trial, including collection of blood samples. Written consent was also obtained from all participants who provided buccal cell samples for genotyping subsequent to completion of the clinical trial. The study protocols for both the clinical trial and the present genotyping study were approved by the institutional review boards at the University of Lisbon and the University of Washington.

### Urinary Mercury Analysis

A urine sample (˜50 ml) was collected from each child at baseline of the clinical trial and at each subsequently scheduled annual visit to the University of Lisbon School of Dental Medicine for dental, neurologic, and neurobehavioral evaluations. Analysis of total mercury (Hg) was performed by continuous flow, cold vapor spectrofluorometry, as previously described (Pingree, S. D., et al. “Effects of 2,3-Dimercapto-1-Propanesulfonic Acid (DMPS) on Tissue and Urine Mercury Levels Following Prolonged Methylmercury Exposure in Rats,” Toxicol. Sci. 61:224-233, 2001). Urinary creatinine concentrations were measured using a standard colorimetric procedure (Sigma #555-A; Sigma-Aldrich, St. Louis, Mo., USA). Urinary Hg (“HgU”) concentrations were calculated as micrograms per gram creatinine (μ/g creatinine). Urinary Hg concentrations were transformed into natural logs after adding one [ln(HgU+1)] and used in this form as a quantitative measure of Hg⁰ exposure for all statistical analyses.

### Statistical Analyses

This study evaluated whether CPOX4 or CPOX5 gene status affected the relationship between Hg⁰ exposure and tests of neurobehavioral functions among children who were evaluated annually from baseline through 7 years of follow-up after initial placement of dental amalgam (Hg) or composite resin tooth fillings (DeRouen, T. A., et al., “Neurobehavioral Effects of Dental Amalgam in Children: a Randomized Clinical Trial,” JAMA 295(15):1784-1792, 2006).

Urinary Hg concentrations measured at each annual behavioral test session were employed as the measure of Hg⁰ exposure. Treatment assignment accounted at most for only 17% of the variation in HgU among boys (Year 2 r²=0.171) and 15% among girls (Year 2 r²=0.154). Thus, instead of the dichotomous assignment to amalgam or composite resin treatment groups as performed in the clinical trial, actual urinary Hg concentrations of study subjects were employed in the present analyses.

Statistical analyses were performed using SPSS Version 19 (IBM® SPSS®, Chicago, Ill., USA). The wide range in ages of subjects at the beginning of the clinical trial (8-12 years) and the changing of specific tests administered to various age groups during the course of the trial, e.g., child versus adult versions of some tests, militated against the propitious use of repeated measures analysis. Therefore, concurrent HgU concentrations and neurobehavioral test performance data acquired from the second year of follow-up (Year 2, where mean HgU reached a peak among both boys and girls in the cohort) were employed to estimate the acute effects of Hg⁰ exposure on performance.

Both maximum and cumulative measures of HgU over the entire study period as well as performance outcomes during the last year of the study (Year 7) were employed to evaluate chronic effects of Hg⁰ exposure.

Acute measures of Hg⁰ exposure were calculated as the natural log of HgU adjusted by 1 (ln [HgU+1]). The natural log best accommodates how exposures are distributed biologically, whereas adding 1 minimizes the influence of very small changes in HgU at very low levels (which could have otherwise dominated the analyses). Maximum HgU was simply the largest value of the acute measure across all study years. Cumulative HgU was calculated as the natural log of the sum of all HgU with 1 added to this summation (ln [(ΣHgU)+1]).

Because the effect of the CPOX4 variant on neurobehavioral performance as affected by Hg⁰ exposure was the principal focus of this study, an analytical protocol reflecting this focus was developed. As a first step, a base model was used that included the measure of Hg⁰ exposure (as defined above), CPOX allelic status (dichotomous as either wild type or het/mut for CPOX4), and their interaction term. In addition, this model included the covariates of age at assessment, race, and non-verbal IQ (determined at baseline). Performances on neurobehavioral tests were each individually evaluated as the outcome variable in separate analyses for boys and girls.

Step two was initiated whenever there was a significant interaction term between Hg⁰ exposure and CPOX allelic status. This finding was taken as evidence of effect modification, and thus analyses of effect between Hg⁰ exposure and test performance were calculated separately among boys or girls genotyped as either CPOX wild type or CPOX4 variant status. This strategy provided a clear description of Hg⁰ dose-response relationships within each genotypic group.

Step three involved evaluating main effects when the interaction term between Hg⁰ exposure and CPOX allelic status when genotyped for CPOX4 was not significant. In this case, the interaction term was dropped from the baseline model, and the main effects of Hg⁰ exposure and CPOX status were evaluated among the full cohort of boys or girls.

This analytical approach was repeated for children genotyped for CPOX5 allelic status.

While illustrative embodiments have been illustrated and described, it will be appreciated that various changes can be made therein without departing from the spirit and scope of the invention. All references cited herein are explicitly incorporated by reference in their entireties.

The embodiments of the invention in which an exclusive property or privilege is claimed are defined as follows:

1. A method for predicting the susceptibility or risk of a young human subject to developing a neurobehavioral deficit associated with mercury exposure, comprising:
(a) performing an assay on a biological sample obtained from a subject to determine the presence or absence of a CPOX4 polymorphism in one or both alleles of the coproporphyrinogen oxidase (CPOX) gene; and
(b) classifying the susceptibility of the subject to developing a neurobehavioral deficit associated with mercury exposure, wherein a subject determined to possess the CPOX4 polymorphism in at least one allele of the CPOX gene is classified as having an increased susceptibility to developing at least one neurobehavioral deficit associated with mercury exposure compared to a subject with no CPOX4 polymorphisms.

2. The method of claim 1, wherein the subject possessing the CPOX4 polymorphism in both alleles of the CPOX gene is classified as having an increased susceptibility to developing at least one neurobehavioral deficit associated with mercury exposure compared to a subject with one or no CPOX4 polymorphisms.

3. The method of claim 1, wherein the at least one neurobehavioral deficit negatively effects performance in at least one neurobehavioral domain selected from the group of domains consisting of attention domain, visual-spatial domain, executive functioning domain, learning and memory domain, and motor domain.

4. The method of claim 3, wherein performance in the attention domain can be determined by a Stroop test, a WAIS III—Digit Span test, or a WMS III—Spatial Span test; wherein performance in the visual-spatial domain can be determined by a Simple Reaction Time test or a WAIS III test; wherein performance in the executive functioning domain can be determined by a Wisconsin Card Sort test or an Adult Trials B test; wherein performance in the learning and memory domain can be determined by a RAVALT test or a WMS III—Visual Reproductions test; and wherein performance in the motor domain can be determined by a WRAVMA—Pegs test or a Finger Tapping test.

5. The method of claim 3, wherein a reduction in performance in at least one neurobehavioral domain in a subject possessing the CPOX4 polymorphism in at least one allele of the CPOX gene and exposed to mercury is greater than the added reductions in performance in the same neurobehavioral domain observed for 1) a subject with no CPOX4 polymorphisms and exposed to mercury, and 2) a subject with the same number of CPOX4 polymorphisms with no exposure to mercury.

6. The method of claim 1, wherein a subject possessing the CPOX4 polymorphism in at least one allele of the CPOX gene has an increased susceptibility to developing a neurobehavioral deficit with exposure to a low level of mercury exposure.

7. The method of claim 5, wherein the low level of mercury exposure is an exposure to mercury in an amount sufficient to result in a detectable urinary mercury level of 5 μg/g creatinine or less.

8. The method of claim 1, wherein the subject possessing the CPOX4 polymorphism in at least one allele of the CPOX gene has an increased susceptibility to developing a neurobehavioral deficit with an acute mercury exposure.

9. The method of claim 1, wherein the subject possessing the CPOX4 polymorphism in at least one allele of the CPOX gene has an increased susceptibility to developing a neurobehavioral deficit with a chronic mercury exposure.

10. The method of claim 1, wherein the subject possessing the CPOX4 polymorphism in at least one allele of the CPOX gene has an increased susceptibility to developing a neurobehavioral deficit within about one month of an mercury exposure.

11. The method of claim 1, wherein the subject is considered to be at risk of exposure to mercury.

12. The method of claim 1, wherein the subject is less than 20 years old.

13. The method of claim 12, wherein the subject is a fetus.

14. The method of claim 1, wherein the subject is male.

15. The method of claim 1, wherein the subject is female.

16. The method of claim 1, wherein the biological sample contains genomic DNA or mRNA.

17. The method of claim 16, wherein the assay comprises amplifying a segment of the CPOX gene from the genomic DNA or mRNA, wherein the segment comprises the nucleic acid residues encoding amino acid position 272 of the corresponding CPOX polypeptide.

18. The method of claim 16, further comprising detecting the presence an adenine (A) to cytosine (C) change in exon 4, relative to the wild type CPOX gene, resulting in an asparagine (Asp, N) to histidine (His, H) substitution at amino acid position 272 of the corresponding CPOX polypeptide.

19. The method of claim 18, wherein the detecting the presence an adenine (A) to cytosine (C) change comprises sequencing the DNA segment or performing a nuclease assay specific for the (A) to cytosine (C) change in the segment.

20. The method of claim 1, further comprising performing an assay on a second biological sample obtained from the subject to determine the exposure status of the subject to mercury.

21. The method of claim 20, wherein the second biological sample comprises urine, blood, serum, plasma, or hair.

22. The method of claim 20, wherein the assay performed on the second biological sample is cold vapor atomic fluorescence spectrometry or inductively coupled plasma mass spectrometry.

23. The method of claim 22, wherein the mercury is assayed from a urine sample, and the concentration is determined per units of creatinine present in the urine.

24. The method of claim 1, wherein a subject determined to have an increased susceptibility of developing at least one neurobehavioral deficit associated with mercury exposure is advised to reduce ongoing or future risk of mercury exposure.


SEQUENCE LISTING (9 sequences)

SEQ ID NO: 1 - DNA, 2728 bp, Homo sapiens
ctttccgcctgtgcagctcgccggctcaatactccgggggtctgggtggggggctcaaacgcagggctctgggacgcaggcactgctccaggtgatagtgcgggaacatggccttgcagctgggcaggctgagctcgggcccctgctggctcgtggcgcggggcggctgcggagggccccgcgcctggtcccagtgcggcggcggagggctccgagcctggtcccagcgcagcgcagccggacgcgtctgccggccccctggcccggctggcacggagcagagccgcgggctggggcacggctcgacgtcgagaggcggcccctgggtggggacagggctggccgcggcgctggcggggttggtggggctggccaccgccgccttcgggcatgtgcagcgggcggagatgttgcctaagacctcggggacgcgggccacttcgctggggaggccggaggaggaggaggatgagctggcccaccgctgcagcagcttcatggccccgcctgtgaccgacctgggcgagctgcgaaggaggccgggcgacatgaagaccaagatggagctgctgattctggagacccaggcccaggtgtgccaggctctggcacaggtagacgggggcgccaacttttctgtggaccggtgggagaggaaggaaggaggtggcggcatcagctgtgtacttcaagatgggtgtgttttcgaaaaggctggggtgagcatttctgttgttcatggaaatctttcagaggaagctgcaaaacaaatgagaagcagaggaaaagttctgaagactaaagatggtaaattgccattttgtgctatgggcgtgagctctgttatccaccccaagaatcctcatgctcctactatccatttcaactacagatactttgaagtagaagaagctgatggcaacaagcagtggtggtttggtggtggatgtgacctcactccaacatacttgaatcaagaagacgctgtccattttcacagaactctgaaggaggcttgtgaccagcatggtccagatctctaccccaaatttaaaaaatggtgtgatgattacttctttatagcccatcgtggagaacggcggggcattggtggtatcttttttgatgatcttgactctccgtccaaggaggaggtgtttcgctttgtacagagctgtgccagggctgtagttccttcttacattccccttgtgaaaaagcactgtgatgactcattcaccccccaggagaagctgtggcagcagctcagaagaggacggtatgtagaatttaatctgctgtatgatcggggcacaaagtttggcctcttcactccaggatccagaattgaaagtatcttgatgtctttacctctaactgcccgatgggagtacatgcattcaccctcagagaattccaaagaagctgaaattctggaagttctacgccatccaagggactgggtgcgttgatgcaggcagaatggctgtgcaggggtttggagggcacacgatgtgtgctccccatgccactggtcggcactttgccactgtgtggcagttacccgtgccttagtcttctccactctgcaccctacctcgtgggcagatgataacatgttttggatgctgtcagtgatgaatggtgagatggcagattgtcagagtcaattgattaaacctcatttatacttctagtgtcattttatatgactagtttacaaaataggacattgagtttccaagtattgagataagggaatataaatagtattatatgtatcaggaaatctctcatcttgtttttgtttcatgtattttttaaagttttcatttgtgccacaaaaatctgtcgtggaatatattttattttcattaattcagtgaagttgagacttcatagtaattttagaatgcaacttgaaggtaaaaattttactttgtcaatactgaagtctctgctgtaatccttatatatctttctccagagaacataatattgtcaaatagatacacatttttctaataggtatttagaagcacttgaaataatctctgcatgtgttacaattcagttatttctgtagtttgtaaactctaaagtgacattactattattttagagatgtctaagttgtaattttgatttttgtggaaccattgtttgttaatgttgggattctctgcacttttgaatgtgaaagcttatatccctgaattctgatacttaagagttttctatttcagacatctctgtgtggaagttgagactaagaataatcctagggatgtcatgaatttaggcaatgtttctcatttggaaaatgaaatgagaaataatttccttctttaaagcaagtatatatagtatgagaaacttggaggcatttcatacacacgtttcttaggaaaatggacacattgaaaatgtcctcttttttatattagagattctgcagctctttgctcttaagagcaaatcacaacaggattcttaatgtatgatttctttgttcatatattatgaatgtattattttatttgcttcgtaataaagtttataaggaagagcatctcatacatatcattatcgtggaacacgttgaatgtttgtgattctgtgtggcctttttggggctggaaaatgtatgaattcttcaactgtcttacaagagatctgctaataaattttattattaaataagactaaaaaaaaaaaaaaaaaaaaa

SEQ ID NO: 2 - DNA, 52 bp, Homo sapiens
  misc_feature (27)..(27): Wherein the N at position 27 can be an A or a C.
gaatcccatgtcttgatttggtaggcnacaagcagtggtggtttggtggtgg

SEQ ID NO: 3 - DNA, 19 bp, Artificial sequence
  Synthetic
aagtccacattagaatccc

SEQ ID NO: 4 - DNA, 20 bp, Artificial sequence
  Synthetic
gttgccttcagaaggaacag

SEQ ID NO: 5 - DNA, 26 bp, Artificial sequence
  Synthetic
accactgcttgttgcctaccaaatca

SEQ ID NO: 6 - DNA, 25 bp, Artificial sequence
  Synthetic
accactgcttgtggcctaccaaatc

SEQ ID NO: 7 - DNA, 26 bp, Artificial sequence
  Synthetic
ccagtaatgctgaatctcaaaagtcc

SEQ ID NO: 8 - DNA, 26 bp, Artificial sequence
  Synthetic
ggacagcgtcttcttgattcaagtat

SEQ ID NO: 9 - DNA, 52 bp, Homo sapiens
  misc_feature (27)..(27): Wherein the N at position 27 can be an A or a G.
tacttctttatagcccatcgtggagancggcggggcattggtggtatctttt